(19) United States
(12) Patent Application Publication
(10) Pub. No.: US 2023/0380955 A1
(43) Pub. Date: Nov. 30, 2023

(54) COMBINATION THERAPY FOR TISSUE REPAIR

(71) Applicant: Miach Orthopaedics, Inc., Westborough, MA (US)

(72) Inventors: Rita PAPARAZZO, Hopkinton, MA (US); Stephen WOHLERT, Westborough, MA (US)

(73) Assignee: Miach Orthopaedics, Inc., Westborough, MA (US)

(21) Appl. No.: 18/201,449

(22) Filed: May 24, 2023

Related U.S. Application Data
(60) Provisional application No. 63/345,401, filed on May 24, 2022.

(51) Int. Cl. A61F 2/08; A61L 27/24; A61L 27/36; A61L 27/56; A61B 17/04

(52) U.S. Cl. CPC A61F 2/0811; A61L 27/24; A61L 27/3616; A61L 27/56; A61L 27/3604; A61B 17/0401; A61L 27/3662; A61F 2002/0882; A61F 2002/0858; A61F 2002/0864; A61F 2002/0852; A61F 2002/0835; A61B 2017/0464; A61B 2017/044; A61B 2017/0409; A61B 2017/0412; A61L 2430/10

(57) ABSTRACT

A system for the repair of a ruptured tissue is provided. Aspects of the invention include a repair device comprising a combination of a scaffold and graft attached by one or more sutures to one or more fixation devices and inserted into a repair site via arthroscopic equipment or an open surgical procedure. The system may be used to treat tissue injuries including ruptured ligaments, tendons, and cartilage.

### CROSS REFERENCE TO RELATED APPLICATIONS

This application claims the benefit of priority of U.S. Prov. Pat. App. Ser. No. 63/345,401, filed May 24, 2022, the contents of which are incorporated herein by reference.

### FIELD OF THE INVENTION

The present disclosure relates generally to systems and methods for the repair of a ruptured tissue utilizing an arthroscopic repair system or an open surgical procedure having a combination of a scaffold implant and a graft.

### BACKGROUND OF THE INVENTION

Intra-articular tissues are difficult to heal after rupture. Similarly, the meniscus and articular cartilage in human joints often do not recover from injuries. Unlike tissues outside of joints that heal by forming a fibrin clot, which eventually transforms into a scar, joint injuries hinder the formation of this clot or cause its quick dissolution. As a result, minor injuries to the knee fail to trigger the formation of a clot, preventing the development of joint arthrosis and stiffness. Synovial fluid in joints naturally prevents clot formation, disrupting the healing process and the formation of a fibrin clot scaffold within the joint or intra-articular tissues.

### SUMMARY OF THE INVENTION

An embodiment of the present disclosure includes a tissue repair system. The tissue repair system includes a repair device. The repair device includes an implant sized and shaped for placement in a repair site of a torn tissue. The implant is compressible and expandable and configured to absorb a repair material. The tissue repair system further includes a graft configured to be coupled to the implant. The tissue repair system further includes at least one implant suture configured to position the implant along or adjacent to a ruptured end of the torn tissue. The tissue repair system further includes a first fixation device configured to couple the at least one implant suture to a first bone. The tissue repair system further includes at least one graft suture configured to position the graft along or adjacent to the implant or the ruptured end of the torn ligament. The tissue repair system further includes a second fixation device configured to couple the at least one graft suture to the first bone.

A further embodiment of the present disclosure includes a method for repairing a ruptured tissue. The method includes inserting a repair device comprising a scaffold and a graft proximate a ruptured end of the torn tissue of a patient. The method further includes securing the scaffold to a suture and a first bone with a first fixation device.

### BRIEF DESCRIPTION OF THE DRAWINGS

The figures are illustrative only and are not required for enablement of the invention disclosed herein.

FIG. 1 is a diagrammatic representation of a tissue repair system according to an embodiment of the present disclosure;

FIG. 2 is a diagrammatic representation of the scaffold shown in FIG. 1;

FIG. 3 is a diagrammatic representation of the graft shown in FIG. 1;

FIG. 4 is a diagrammatic representation of a repair device according to an embodiment of the present disclosure;

FIG. 5 is a diagrammatic representation of a repair device according to an embodiment of the present disclosure;

FIG. 6 is a diagrammatic representation of the arthroscopic repair system according to an embodiment of the present disclosure;

FIG. 7 is a schematic depicting the tissue repair system being utilized for ligament repair in the ACL;

FIG. 8 is a schematic depicting the repair device shown in FIG. 8 being inserted into a repair site;

FIG. 9 is a schematic depicting the tissue repair system shown in FIGS. 7-8 being fully inserted in the repair site;

FIG. 10 is a schematic depicting the tissue repair system shown in FIGS. 7-9 being secured in the repair site;

FIG. 11 is a schematic depicting the tissue repair system being utilized for tendon repair in the Achilles tendon;

FIG. 12 is a schematic depicting the tissue repair system shown in FIG. 12 being inserted into the repair site;

FIG. 13 is a schematic depicting the tissue repair system being utilized for cartilage repair in the elbow;

FIG. 14 is a schematic depicting the tissue repair system shown in FIG. 14 being inserted into the repair site; and

FIG. 15 is a schematic showing the ruptured cartilage shown in FIGS. 14-15 being repaired by the tissue repair system.

### DETAILED DESCRIPTION OF THE INVENTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, left, right, above, below and diagonal, are used with respect to the accompanying drawings. Such directional terms used in conjunction with the following description of the drawings should not be construed to limit the scope of the present disclosure in any manner not explicitly set forth.

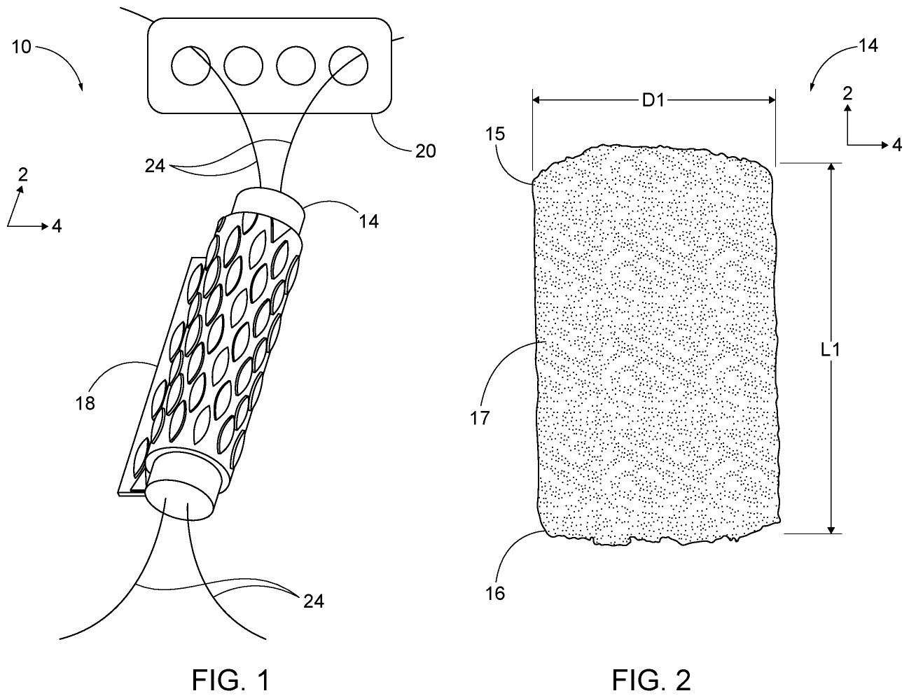

Referring to FIG. 1, the system 1 includes a repair device 10 comprising a scaffold 14 and a graft 18, each of which are configured to be inserted at a repair site in order to repair the ruptured or damaged tissue. Tissue here may include a ligament (for example, an anterior cruciate ligament or “ACL”), a tendon (for example, an Achilles tendon), or cartilage (for example, elbow cartilage). The system 1 further includes one or more fixation devices 20 configured to secure the repair device 10 in place. The system 1 further includes one or more sutures 24 coupled to the scaffold 14 and the graft 18 to form the repair device 10 and configured to connect the repair device 10 to the repair site as described hereinafter.

The repair device 10 is configured to heal or repair tissue. More specifically, the repair device 10 allows the subject's body to develop a network of capillaries, arteries, and veins at the tissue tear or rupture. Well-vascularized connective tissues heal as a result of migration of fibroblasts into the repair device 10. The methods and systems of the present disclosure establish a link between the damaged tissue, either by encircling the torn tissue or connecting with it, fostering the mending process of the ruptured or torn tissue while preserving its integrity and structure. The scaffold may function either as an insoluble or biodegradable regulator of cell function or as a delivery vehicle of a supporting structure for cell migration or synthesis, and may provide a network or structure to facilitate cell in growth and vascularization. The graft may provide additional mechanical stability during the healing phase.

The present disclosure provides a repair device 10 comprising a three-dimensional (3-D) scaffold 14 and graft 18 for repairing a ruptured or torn tissue. The scaffold 14 and the graft 18 are combined to provide a connection between the ruptured or torn portions of the tissue and fibers, or form around the torn tissue, after injury, and encourages the migration of appropriate healing cells to form scar and new tissue. The scaffold 14 is a bioengineered substitute for a clot and is implanted with the graft 18. The repair device 10 is thus implanted between the ruptured or torn portions of the tissue, or wrapped around the tissue, or placed adjacent to the ruptured or torn tissue. In other embodiments, the repair device 10 is capable of forming a connection between bone, or forming around the torn tissue such that the integrity and structure of the tissue is maintained. This repair device 10 is therefore designed to stimulate cell proliferation and extracellular matrix production in the gap between the ruptured ends of the tissue or the tear in the tissue, thus facilitating healing and regeneration.

As used herein, the injury is a torn tissue including a torn or ruptured ligament, tendon, or cartilage. A torn tissue can manifest as either a partial tear or a complete tear. A partial tear occurs when a portion of the tissue is damaged, yet it remains connected. The extent and shape of the tear can vary. On the other hand, a ruptured tissue, also known as a complete tear, involves the complete severing of the tissue, resulting in two distinct ends. These ends may have similar or different lengths. In some cases, a tissue stump may be formed at one end. For example, in complete anterior cruciate ligament (“ACL”) tears, the ligament stumps include a tibial stump connected to the tibia and a femoral stump connected to the femur.

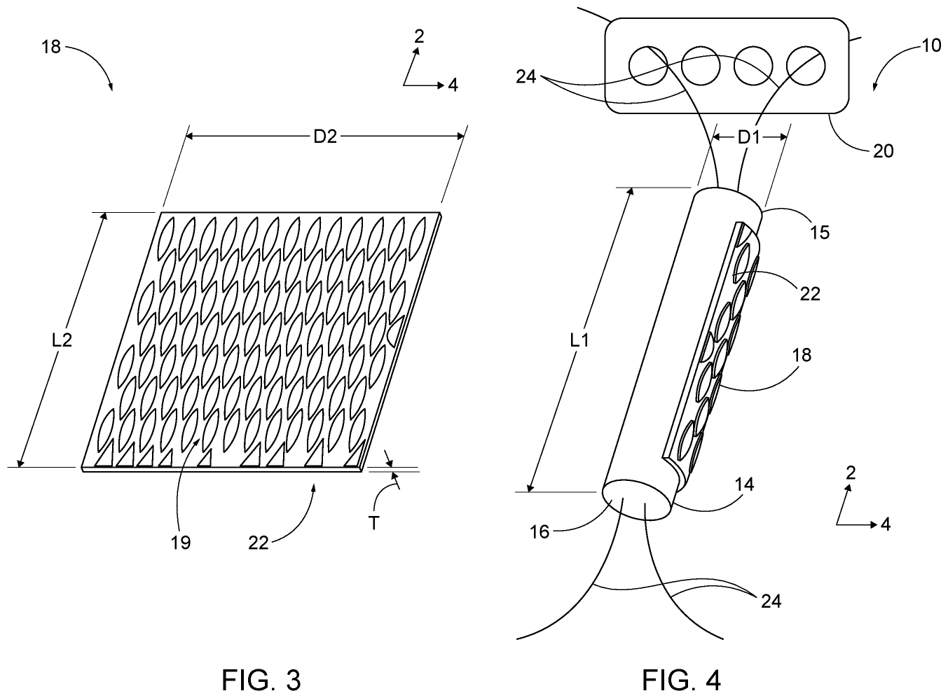

Referring to FIG. 2, the scaffold 14 is a compressible and biocompatible implant that is configured to absorb fluid, such as blood and/or blood components. The scaffold may be a compressible and expandable, biodegradable, porous material that has some resistance to degradation by fluids in the tissue repair site, (for example, synovial fluid). In the illustrated embodiment, the scaffold 14 is a collagen implant comprising a self-assembly of interconnected collagen fibers that does not include any cross-linking agents. The collagen itself may include a collagen-glycosaminoglycan (“GAG”) copolymer comprised of soluble type I collagen. In one example, the scaffold 14 has a collagen content of greater than 400 mg/g, a GAG content of greater than 100 μg/g, a DNA content of less than 50,000 ng/g, a phospholipid count of less than 3,000 μM/g and a pepsin content of less than 12.5 mg/g. While the scaffold 14 may be Type I collagen, the scaffold may include other collagen types. Preferably, the collagen is soluble, e.g., acidic or basic. For example, in another embodiment, the collagen may be type II, III, IV, V, IX or X.

The scaffold may have a size and shape selected complement to the specific anatomical tissue present, as further explained below. In one example, the scaffold 14 may be a generally elongated structure having a first end 15, a second end 16 opposite the first end 15, and a sidewall 17 that extends from the first end 15 to the second end 16. In this manner, the scaffold 14 is cylindrical in shape having a length L1, which extends from the first end 15 to the second end 16 along a central axis A. The length L1 may range between 15 mm and 35 mm. In one example, the length L1 is at least 25 mm. The scaffold 14 may have a cross-sectional dimension D1, which is perpendicular to the length L1, that ranges between 10 mm and 30 mm. In one example, the cross-sectional dimension is between 20 mm and 25 mm. In another example, the cross-sectional dimension D1 is about 22 mm. Thus, in a generally elongated scaffold, the length L1 is greater than the cross-sectional dimension D1. A cylindrically shaped scaffold is suitable for use with ACL procedures, as described below. However, the size and shape of the scaffold 14 can vary as needed for other anatomies and tissue sites.

The scaffold 14 is hydrophilic and capable of absorbing plasma, blood, other body fluids, liquid, hydrogel, or other material the scaffold either comes into contact with or is added to the scaffold. The scaffold 14 may be referred to hereinafter as a collagen scaffold, sponge, or collagen sponge.

In the illustrated embodiment, the scaffold 14 is treated with a repair material prior to insertion into a repair site. As illustrated, the repair material is blood. Specifically, the scaffold 14 is treated with 5-10 mL of autologous blood. The repair device 10 may be either soaked in the repair material or the repair material is injected into the repair device 10 prior to or during implantation into the repair site. In other examples, the repair material may be an autologous or allogeneic blood composition, plasma or other fluids either present within the repair site, added to the scaffold 14, or added into the repair site.

While a cylindrical shape is shown as suitable for certain tissue repairs, the scaffold 14 may be any shape that is useful for implantation into and repair of tissue. The scaffold 14, for instance, may be tubular, semi-tubular, a flat planar sheet, or a flat sheet rolled into a tube so as to define a hollow cavity. Other shapes suitable for the scaffold of the device as known to those of ordinary skill in the art are also contemplated in the invention.

In addition, in alternative embodiments, the scaffold may include additional components to aid in healing, cellular ingrowth and vascularization. Such additional components may include proteins, including, but not limited to, hormones, cytokines, growth factors, clotting factors, anti-protease proteins (e.g., alpha1-antitrypsin), angiogenic proteins (e.g., vascular endothelial growth factor, fibroblast growth factors), antiangiogenic proteins (e.g., endostatin, angiostatin), and other proteins that are present in the blood, bone morphogenic proteins (BMPs), osteoinductive factor (IFO), fibronectin (FN), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, transforming growth factor (TGF-beta), insulin-like growth factor (IGF-1), platelet derived growth factors (PDGF), fibroblast growth factors (FGF, bFGF, etc.), and periodontal ligament chemotactic factor (PDLGF), for therapeutic purposes. A lyophilized material is one that is capable of swelling when liquid, gel or other fluid is added or comes into contact with it.

The scaffold 14 may be compressed prior to or during implantation into a repair site. A compressed scaffold allows for the scaffold to expand within the repair site. The scaffold 14 may be lyophilized and/or compressed when placed in the repair site and expanded once in place. The expansion of the scaffold 14 may occur after contact with blood or other fluid in the repair site or added to the repair site.

In another embodiment, the scaffold 14 may be saturated or coated with a gel or hydrogel repair material prior to implantation into a repair site. Coating or saturation of the scaffold 14 may ease implantation into a relatively undefined defect area as well as help to fill a particularly large defect area. In a preferred embodiment, the scaffold 14 is treated with hydrogel. Examples of scaffolds and repair materials that may be used according to the present disclosure are found in U.S. Pat. No. 6,964,685 and U.S. Patent Application Nos. 2004/0059416 and 2005/0261736, the entire contents of each are herein incorporated by reference.

The biologic properties of cell infiltration rate and scaffold degradation may also be altered by varying the pore size, degree of cross-linking, and the use of additional proteins, such as glycosaminoglycans, growth factors, and cytokines in the scaffold 14. In addition, collagen-based biomaterials can be manufactured from a patient's own skin, thus minimizing the antigenicity of the implant. However, preferable collage scaffolds do not include any cross-linking agents.

Referring to FIG. 3, a graft 18 is used to provide some mechanical stability and can serve as a support for the scaffold. In the embodiment shown, the graft 18 is a flat, sheet-like structure configured to wrap partially or completely around the scaffold 14. The graft 18 has a first surface 19, a second surface 22 opposing the first surface 19, a thickness T that extends from the first surface to the second surface, a length L2 that extends along a longitudinal direction 2 and a cross-sectional dimension D2 that extends along a lateral direction 4 that is perpendicular to the longitudinal direction 2. The length L2, width D2, and thickness T are perpendicular to each other when in the flat state. The length L2 may range between 15 mm and mm. In one example, the length L2 is about 25 mm. The width D2 may range between 0.05 mm and 35 mm. In one example, the width D2 is about 35 mm. The thickness T may range between 0.01 mm and 0.05 mm. In one example, the thickness T is about 0.01 mm. The length L2 may be less than the length L1, which allows exposure of the ends 15, 16 of the scaffold and may help facilitate attachment to the bone or other tissue as needed. As illustrated, the graft length L is greater than the graft width W. In alternative embodiments, the dimensions of the graft 18 can be defined during manufacturing to any particular size. The graft 18 may be folded or rolled to achieve a desired length and width.

Several types of grafts are available for use in tissue reconstruction and repair. In the illustrated embodiment, the graft 18 may be an autologous graft or autograft that is harvested from the patient, for example a patellar bone-tendon-bone graft, or a hamstring graft. Alternatively, the graft 18 may include one or more xenografts, allografts, isografts, or synthetic polymer grafts either alone or in any combination. In one embodiment, the graft 18 is an allograft. Allografts include ligamentous tissue harvested from cadavers and appropriately treated and disinfected, and preferably sterilized. In another embodiment, the graft 18 is a xenograft. Xenografts include harvested connective tissue from animal sources such as, for example, porcine tissue. Typically, the xenografts must be appropriately treated to eliminate or minimize an immune response. In yet another embodiment, the graft 18 is a synthetic graft. Synthetic grafts include grafts made from synthetic polymers and/or polymeric filaments, such as polyurethane, polyethylene, polyester and other conventional biocompatible bioabsorbable or nonabsorbable polymers and composites, such as the scaffolds described herein. Material for synthetic grafts may include, but are not limited to Supramid®, Teflon® or Dacron®, Proplast®, carbon fiber graft, ABC graft, Kennedy-LAD®, Trevia, Leeds-Keio, Gore-Tex®, PDS®, EULIT®, and Polyflex® or LARS®.

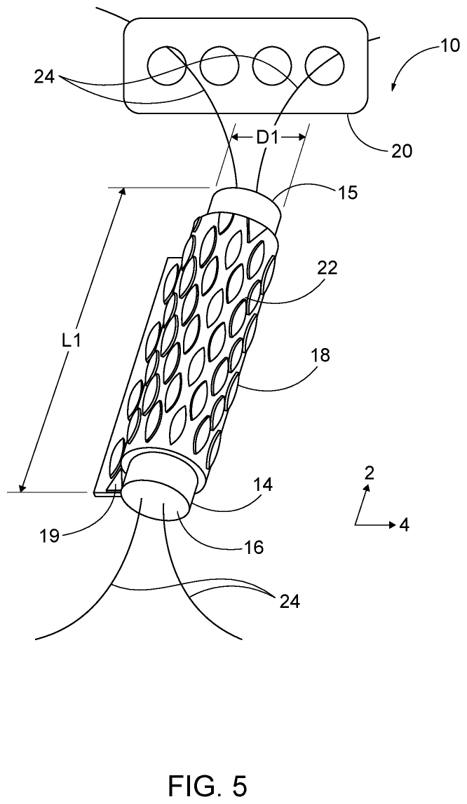

Examples of the combination of the scaffold 14 and the graft 18 to create the repair device 10 is depicted in FIGS. 4 and 5. Referring to FIG. 4, in one illustrated embodiment, the graft 18 is attached to the scaffold 14 such that the graft 18 is coupled to only a portion of the scaffold 14. Thus, in the illustrated embodiment, the graft 18 may be positioned along or adjacent to the scaffold 14. The scaffold 14 may be attached to the graft 18 via one or more sutures 24. In alternative embodiments, the scaffold 14 may be attached to the graft via other mechanisms known in the art.

Referring to FIG. 5, in another illustrated embodiment, the graft 18 is wrapped around the scaffold 14 such that the graft 18 surrounds the scaffold 14. In an alternative embodiment, the graft 18 may be wrapped around a portion of the scaffold 14. or a majority of the scaffold 14. In the illustrated embodiment, the graft 18 may be wrapped around the scaffold 14 via sutures 24 to hold the repair device 10 in place inside the patient's body. In alternative embodiments, the graft 18 may be wrapped around the scaffold 14 via other mechanisms known in the art.

In one example, the scaffold 14 may be attached between the femoral insertion point and the tibial insertion point and may sit conjunctive to the graft 18. In another example, the graft 18 may be inserted or pushed through the scaffold 14.

For example, In another embodiment, The scaffold 14 and graft 18 may be attached to a one or more sutures 24 and a one or more fixation devices 20. The one or more fixation devices may, as shown in FIGS. 1B and 1C, be attached to the one or more sutures 24 through an eyelet 10 of the one or more fixation devices 20. In this configuration, the one or more fixation devices 20 is attached into a bone. The bone may be either the femur 4 or the tibia 6.

In one embodiment, both the scaffold 14 and the graft 18 are pretreated with repair material. The scaffold 14 and the graft 18 may be soaked in a repair material prior to or during implantation into the repair site. Repair material includes, but is not limited to, a gel, for example a hydrogel, or a liquid, or any material capable of forming an aqueous material, a suspension, or a solution that is injectable into the scaffold and can aid in tissue repair or growth. In one example, the repair material is blood. In alternative embodiments, the repair material may include a composition including plasma, platelets, growth factors, antibiotics, thrombin, stem cells, a genetically altered fibroblast, platelets, plasma, extracellular proteins, and/or a cell media supplement. The additional repair materials may be added to affect cell proliferation, extracellular matrix production, consistency, inhibition of disease or infection, tonicity, cell nutrients until nutritional pathways are formed, and pH of the repair material. All or a portion of these additional materials may be mixed with the repair material before or during implantation, or alternatively, the additional materials may be implanted proximate to the defect area after the repair material is in place.

In certain embodiments, platelets may be obtained as platelet rich plasma (PRP). In a non-limiting example, platelets may be isolated from a subject's blood using techniques known to those of ordinary skill in the art. As an example, a blood sample may be centrifuged at 700 rpm for 20 minutes and the platelet-rich plasma upper layer removed.

Referring to FIGS. 4 and 5, the system 1 includes one or more fixation devices 20, such as a first fixation device 20A and a second fixation device 20B. The first fixation device is configured to indirectly couple the scaffold 14 to a repair site via one or more sutures 24. The second fixation device 20B is configured to indirectly couple the graft 18 to the repair site via one or more sutures 24. The first and second fixation devices 20A, 20B may be similar to each so only one is described below. Each fixation device 20 is a device capable of insertion into the repair site and help forms a stable attachment of the repair device to the surrounding tissue. In some instances, the one or more fixation devices 20 is capable of being removed from the repair site if desired.

The fixation device 20 is further configured to connect to the one or more sutures 24. In one example, the fixation device may be an elongated plate with a plurality of eyelets or openings that extends through the plate. The plate has a length that is greater than its width and thickness. This profile allows the fixation device 20 to be inserted through cannulas and bone tunnels as needed. The openings are suitable for allowing the one or more sutures 24 to be threaded through the one or more fixation devices 20 and fastened into position in the repair site. The one or more openings 21 may be oval or round and may be of any size suitable to allow the one or more sutures 24 to pass through and be held within the one or more openings 21. An example of such a fixation device is known as Endobutton.

The fixation devices may have other configurations. The fixation device 20 may include, but is not limited to, a screw, a barb, an anchor, a helical anchor, a staple, a clip, a snap, a rivet, or a crimp-type anchor. The body of the one or more fixation devices 20 may be varied in length. Examples of fixation devices, include but are not limited to, IN-FAST™ Bone Screw System (Influence, Inc., San Francisco, CA), IN-TAC™ Bone Anchor System (Influence, Inc., San Francisco, CA), Model 3000 AXYALOOP™ Titanium Bone Anchor (Axya Medical Inc., Beverly, MA), OPUS MAGNUM® Anchor with Inserter (Opus Medical, Inc., San Juan Capistrano, CA), ANCHRON™, HEXALON™, TRINION™ (all available from Inion Inc., Oklahoma City, OK) and TwinFix AB absorbable suture anchor (Smith & Nephew, Inc., Andover, MA).

In the illustrated embodiment, the first fixation device 20A is coupled to a first bone at the repair site and indirectly attaches the scaffold 14 to the first bone via the one or more sutures 24 attached to the repair device 10. The first fixation device 20A helps to position and secure the repair device 10 to the first bone at the repair site. The first fixation device 20A may be inserted and anchored to the first bone.

The second fixation device 20B is coupled to a second bone at the repair site and indirectly attaches the graft 18 to the first bone via the one or more sutures 24 attached to the repair device 10. The second fixation device 20B helps to position and secure the repair device 10 at the repair site. The second fixation device 20B may be inserted and anchored to the second bone.

In one embodiment, the system utilizes only one fixation device 20 attached to the scaffold 14 and the graft 18 and further connected to the repair site. In an alternative embodiment, the system utilizes more than one fixation device 20, and fixation devices 20 may be attached directly to the repair device 10 via the one or more sutures 24. In this configuration, the one or more fixation devices 20 are swaged directly onto the scaffold 14 and/or the graft 18.

The one or more fixation devices 20 may be composed of a non-degradable material, such as metal, stainless steel, CoCrMo alloy, or Nitinol alloy, or polymeric materials. The one or more fixation devices 20 is preferably bioabsorbable such that the subject is capable of breaking down the one or more fixation devices 20 and absorbing it.

Continuing with FIGS. 4 and 5, in the illustrated embodiment, the one or more fixation devices 20 are attached to the repair device 10 via the one or more sutures 24. The one or more sutures 24 are passed through the eyelet 21 of the one or more fixation devices 20 and held within the one or more openings 21 such that the one or more fixation devices 20 are attached to the repair device 10 by the one or more sutures 24. In the illustrated embodiment, the one or more sutures 24 have two free ends, a first end 26 and a second end 28 that emerge from the repair device 10. In the illustrated embodiment, at least one additional suture is configured to position the graft 18 along or adjacent to the scaffold 14. However, in other embodiments, only one suture is needed to position the graft 18 along or adjacent to the scaffold 14.

In the illustrated embodiment, the one or more sutures 24 are bioabsorbable, such that the subject is capable of breaking down the one or more sutures 24 and absorbing it. The one or more sutures 24 are also synthetic such that the suture may not be from a natural source. In other embodiments, the one or more sutures 24 may be permanent such that the subject is not capable of breaking down the suture and the one or more sutures 24 remains in the subject. The one or more sutures 24 may be rigid or stiff, or may be stretchy or flexible. Examples of sutures include, but are not limited to, VICRYL™ polyglactin 910, PANACRYL™ absorbable suture, ETHIBOND® EXCEL polyester suture, PDS® polydioxanone suture and PROLENE® polypropylene suture. Sutures are available commercially from manufacturers such as MITEK PRODUCTS division of ETHICON, INC. of Westwood, Mass.

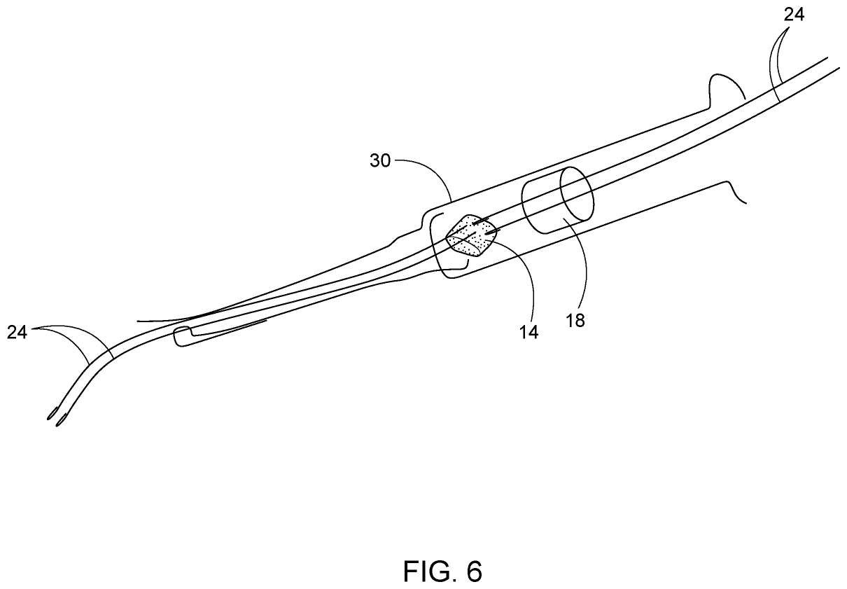

Referring to FIG. 6, an arthroscopic equipment 30 is configured to insert the one or more sutures 24 through the scaffold 14 and the graft 18. The arthroscopic equipment 30 is configured to contain the scaffold 14, the graft 18, and the one or more sutures 24. During implantation, the arthroscopic equipment 30 introduces the scaffold 14, the graft 18, and the one or more sutures 24 into the tissue defect. In the illustrated embodiment, the arthroscopic equipment 30 introduces the repair device by pushing or releasing the repair device from the container into the repair site.

The arthroscopic equipment 30 is further configured to position the scaffold 14 and the graft 18 in the repair site. The arthroscopic equipment 30 includes an elongated delivery member 31. The elongated delivery member 31 includes a channel that extends from a proximal end to a distal end of the elongated delivery member 31. The elongated delivery member 31 is sized and shaped to contain the scaffold 14 and the graft 18 attached to the one or more sutures 24 in the channel. At least a portion of the elongated delivery member 31 is further sized and shaped to be capable of being inserted into the repair site.

In the illustrated embodiment, the arthroscopic equipment 30 is a syringe. The syringe may hold the one or more sutures 24 and the scaffold 14 and the graft 18 in place within the elongated delivery member 31 of the syringe. The syringe may include a plunger 32 configured to push the one or more sutures 24, the scaffold 14 and the graft 18 into a repair site such that the scaffold 14 and the graft 18 are positioned along the one or more sutures 24 and adjacent to at least one ruptured end of the tissue. In alternative embodiments, the arthroscopic equipment 30 may include a cannula, a container, and a pressure pump. In another embodiment, the arthroscopic equipment 30 may further include a guiding suture that extends out of the distal end of the elongated delivery member, where the guiding suture is configured to pull and position the one or more sutures 24 and the scaffold 14 into the repair site. In yet another embodiment, the system may be inserted into the repair site without the use of arthroscopic equipment and instead through an open surgical procedure.

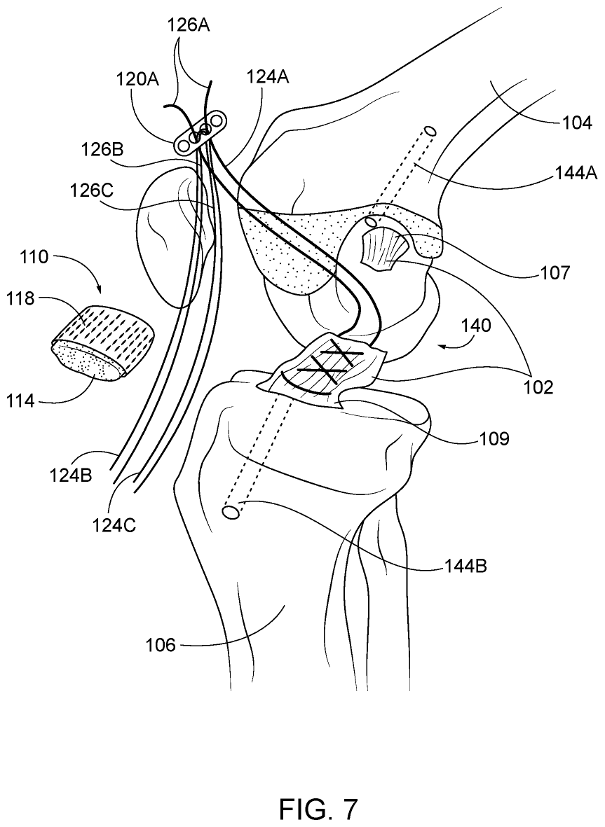

Referring to FIGS. 7-10, aspects of the invention relate to methods of repairing a ruptured or torn ligament, such as an ACL, at a repair site 140. In some embodiments, the scaffold 114, the graft 118, and the one or more sutures 124 are inserted into the repair site 140 of the ruptured or torn ligament 102 via the arthroscopic equipment 130.

The repair site 140 is the area around a ruptured or torn ligament 102 into which a device may be inserted. The scaffold 114 and the graft 118 may be inserted into the repair site 140 during surgery via the arthroscopic equipment 130. The scaffold 114 is expandable and can either fill the repair site 140 with the graft 118 or partially fill the repair site 140 with the graft 118. The scaffold 114 can partially fill the repair site 140 when inserted and expand to fill the repair site 140 in the presence of blood, plasma or other fluids either present within or added into the repair site 140.

In the illustrated embodiment, the scaffold 114 and the graft 118 are coupled to form the repair device 110 and the repair device 110 is attached directly or indirectly to bone and contacts the ruptured or torn ligament 102. In another embodiment, the repair device 110 may form around the ruptured or torn ligament 102 at the repair site 140. For example, in one embodiment, the repair device 110 is wrapped around the ligament 102, in another embodiment, the repair device 110 is positioned behind the ligament such that the ligament is held within the repair device 110. In yet another embodiment, the repair device 110 may be a “Chinese finger trap” design where one end is placed over a stump of a ruptured ligament and the second end is placed over the other end of the ruptured ligament.

In an alternative embodiment, the graft 118 is coupled to the one or more sutures 124 and the one or more fixation devices 120 to form a graft construct with desirable dimensions. The graft 118 is advanced into a first bone and coupled to the one or more fixation devices 120 on the first bone. The graft 118 is further coupled to a second bone via the one or more fixation devices 120 and tensioned in extension. The graft 118 is subsequently coupled to the scaffold 114.

An example of a ruptured anterior cruciate ligament is depicted in FIG. 7. The anterior cruciate ligament (ACL) 102 is one of four strong ligaments that connects the bones of the knee joint. The function of the ACL is to provide stability to the knee and minimize stress across the knee joint. It restrains excessive forward movement of the lower leg bone, the tibia 106, in relation to the thigh bone, the femur 104, and limits the rotational movements of the knee.

As shown in FIGS. 7-10, the anterior cruciate ligament 102 is ruptured such that it no longer forms a connection between the femur bone 104 and the tibia bone 106. The resulting ends of the ruptured ACL 102 may be of any length. The ends may be of a similar length, or one end may be longer in length than the other. The end on the femur 104 includes the femoral ACL stump 107. The end on the tibia 106 includes a tibial stump 109. In some instances, it is believed that a repair is desirable when the tibial stump length SL is less than about 75% of the effective ligament length LL but greater than 5% of a total length LL of the ACL. The total length of the ACL is considered to be the length of ligament from femoral footprint to the tibial footprint along a linear axis.

The knee joint includes tibial spines on the tibia 106 and the intercondylar notch of the femur 104. In some instances, the methods as described herein may include performing a notchplasty of the intercondylar notch of the femur to provide space for larger ligament to form after surgical repair using a scaffold. Such a notchplasty improves the size of the healing ligament, specifically resulting in a larger cross-sectional area of the ligament. As the mechanical strength of a ligament, and subsequently its ability to maintain the distance between the femur and tibia, is directly correlated with its cross sectional area, enlarging the notch with a notchplasty can help make a stronger repaired ACL and has been found by the inventors to be beneficial in ACL repair using a scaffold as described in the present disclosure.

Aspects of the invention provide methods of repairing the ruptured ligament 102 involving drilling one or more holes 144 at or near the repair site 140 of the ruptured ligament 102. A bone at or near a repair site is one that is within close proximity to the repair site and can be utilized using the methods and devices of the invention. For example, the bone at or near a repair site of a torn anterior cruciate ligament is the femur 104 bone and/or the tibia 106 bone. The hole 144 can be drilled into a bone using a device such as a Kirschner wire (for example a small Kirschner wire) and drill, or microfracture pics or awls. One or more holes may be drilled into a bone surrounding the repair site 140 to promote bleeding into the repair site 140. The repair can be supplemented by drilling holes into the surrounding bone to cause bleeding. Encouraging bleeding into the repair site may promote the formation of blood clots and enhance the healing process of the injury.

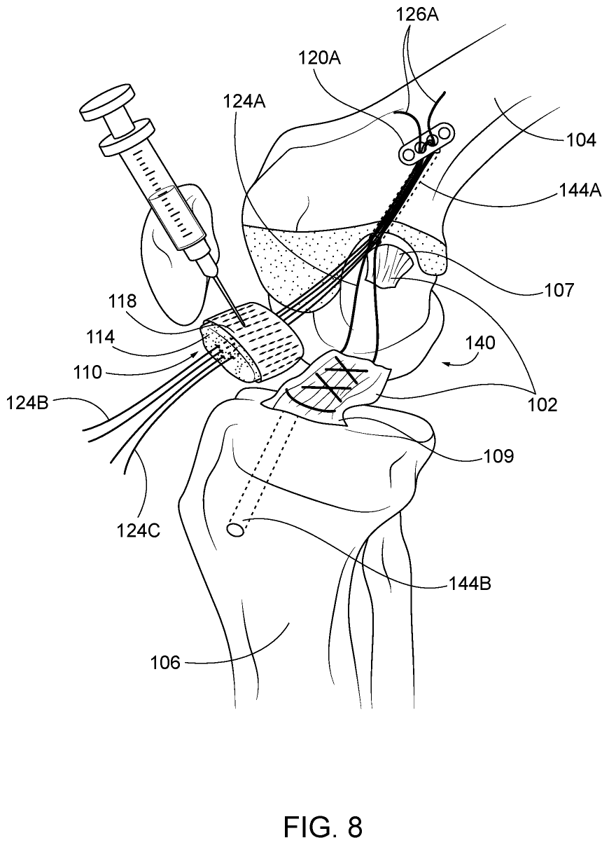

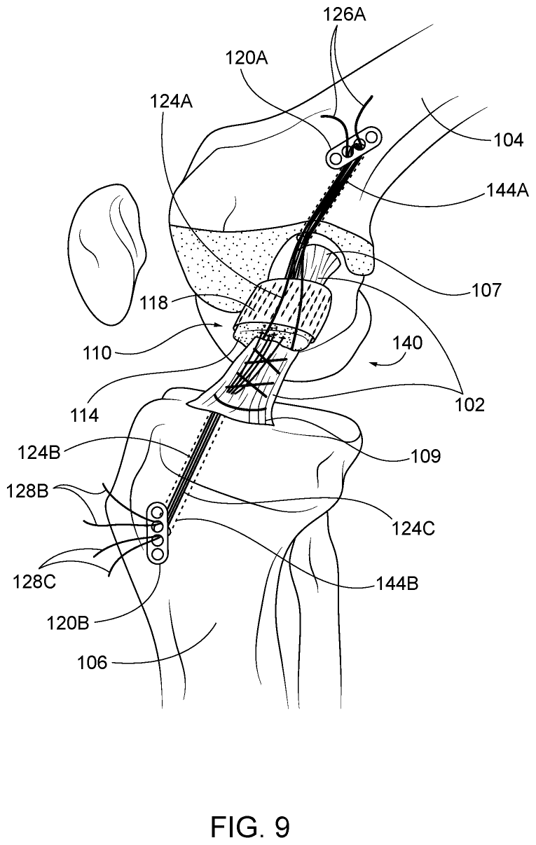

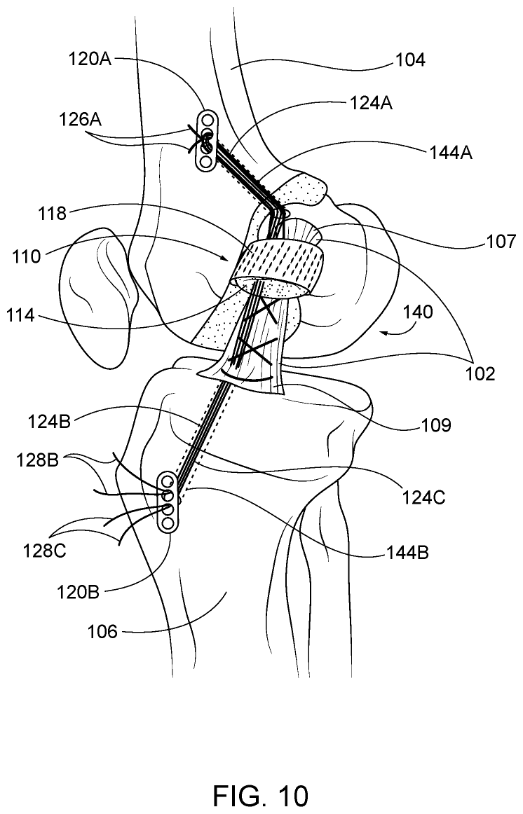

In FIG. 7, holes 144A, 144B are drilled into the femur 104 and the tibia 106, respectively, at the repair site 140. The hole 144A may be additionally referred to hereinafter as the femoral tunnel 144A and the hole 144B may be additionally referred to hereinafter as the tibial tunnel 144B A first suture 124A is placed through the tibial stump 109 using a whip-stitch. The first suture 124A is attached via the first end 126A to a first fixation device 120A. A second suture 124B and a third suture 124C are coupled to the fixation device 120A at respective first ends 126B, 126C. The fixation device 120A is subsequently passed through the femoral tunnel 144A and coupled to the femur 104. In FIG. 8, the repair device 110 is loaded onto the second and third sutures 124B, 124C. The repair device 110 is then injected with repair material as described. The repair device 110 and the second and third sutures 124 may be inserted into the repair site 140 via the arthroscopic equipment 130. In FIG. 9, the free ends 128B, 128C of the second and third sutures 124B, 124C are passed through the tibial tunnel 144B and are coupled to a second fixation device 120B coupled to the tibia 106. The repair device 110 is then positioned between the two ends of the torn ACL 2. In FIG. 10, the knee is extended and the sutures 124A, 124B, 124C, and the fixation devices 120A, 120B are secured.

In another embodiment, the repair device 110 may be indirectly coupled to the first and second fixation devices 120A, 120B and held in position in the repair site 140 by additional sutures 124. In addition, in another embodiment, any of the first, second, or additional sutures 124A, 124B, . . . 124n may be attached to one or both ends of a ruptured ligament 102 by their first ends 126A, 126B, . . . 126n and/or their second ends 128A, 128B, . . . 128n. Furthermore, in another embodiment, additional fixation devices 120 and sutures 124 may be directly or indirectly attached to either the tibia bone 6 or the femur bone 104 to secure the scaffold 114 and the graft 118 in position. In alternative embodiments, the scaffold 114 and the graft 118 may be attached to the femur bone 104 directly or indirectly. For example, the graft 118 may be attached to the scaffold 114 via sutures or via another mechanism. In another embodiment, the graft 118 may be positioned along or adjacent to the scaffold 113. In one example, the scaffold 114 may be attached between the femoral insertion point and the tibial insertion point and may sit conjunctive to the graft 118. In another example, the graft 118 may be inserted or pushed through the scaffold 114.

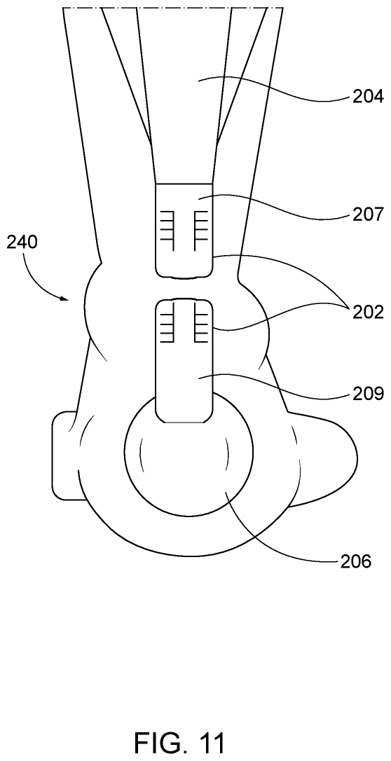

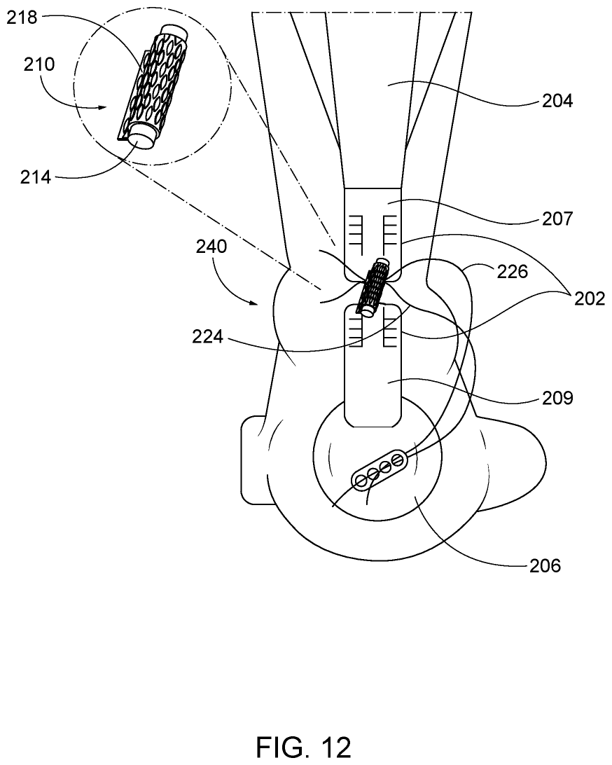

Referring to FIGS. 11 and 12, aspects of the invention relate to methods of repairing a ruptured or torn tendon 202, at a repair site 240, involving a repair device 210 comprising a scaffold 214 and a graft 218, one or more fixation devices 220, and one or more sutures 224. In some embodiments, the scaffold 214, the graft 218, and the one or more sutures 224 are inserted into the repair site 240 of the ruptured or torn tendon 202 via arthroscopic equipment 130 (not depicted).

The repair site 240 is the area around a ruptured or torn tendon 202 into which a device may be inserted. The repair device 210 may be inserted into the repair site 240 during surgery via the arthroscopic equipment 130 using techniques known to those of ordinary skill in the art. The scaffold 214 is expandable and can either fill the repair site 240 with the graft 218 or partially fill the repair site 240 with the graft 218. The scaffold 214 can partially fill the repair site 240 when inserted and expand to fill the repair site 240 in the presence of blood, plasma or other fluids either present within or added into the repair site 240.

An example of a ruptured calcaneal (“Achilles”) tendon is depicted in FIG. 12. The Achilles tendon 202 is a vital structure that connects the calf muscle 204 to the calcaneus 206. The Achilles tendon helps in transmitting the force from the calf muscle 204 to the foot, enabling movements such as walking, running, and jumping. It acts as a stabilizer for the ankle joint, preventing excessive movement and providing support during various activities. Additionally, the Achilles tendon limits rotational motions of the foot and ankle, contributing to overall joint stability and minimizing stress on the surrounding structures.

As shown in FIGS. 11 and 12, the Achilles tendon 202 is ruptured such that it no longer forms a connection between the calf muscle 204 and the calcaneus 206. The resulting ends of the ruptured tendon 202 may be of any length. The ends may be of a similar length, or one end may be longer in length than the other. The end on the calf muscle 204 includes the calf stump 207. The end on the calcaneus 206 includes a calcaneal stump 209. The total length of the tendon is considered to be the length of ligament from calf footprint to the calcaneal footprint along a linear axis.

Prior to insertion of the repair device 210, the affected extremity is prepared and draped in the standard sterile fashion. A tourniquet may be used if indicated. The ruptured tendon 202 is identified and defined, and the tissue ends are pretreated, either mechanically or chemically. The one or more sutures 224 is connected to one or more fixation devices 220.

Prior to entering the repair site 240, the arthroscopic equipment 130 attaches the one or more sutures 24 to the repair device 210. The repair device 210 is treated with a repair material. In one embodiment, the repair device 10 may also be pre-treated in antibiotic solution prior to implantation.

In the illustrated embodiment, the one or more sutures 224 is then connected to the ruptured end of the tendon 202 at the first end 226. In one embodiment, the one or more sutures 224 is placed through the ruptured end of the ligament 202 using a whip-stitch. The one or more fixation devices 220 is passed through the calcaneus 206, carrying the one or more sutures 224. The one or more fixation devices 220 and the one or more sutures 224 is attached to the bone 206.

The arthroscopic equipment 130 (not depicted) positions the repair device 210 along the one or more sutures 224 between the ruptured ends of the ligament 202. In alternative embodiments, the arthroscopic equipment 130 positions the repair device 210 directly or indirectly onto the calf muscle 204 and/or the calcaneus 206. The present disclosure may be used by insertion through an open incision. The repair device is compressible to allow introduction through arthroscopic portals, incisions and equipment.

The repair device 210 is then bonded to the surrounding tissue using the methods described herein. This can be done by the addition of a chemical agent or a physical agent such ultraviolet light, a laser, or heat. The repair device 210 may be reinforced by placement of additional sutures or clips. The arthroscopic portals is closed, and a sterile dressing placed. The post-operative rehabilitation is dependent on the type and size of lesion treated, and the tissue involved.

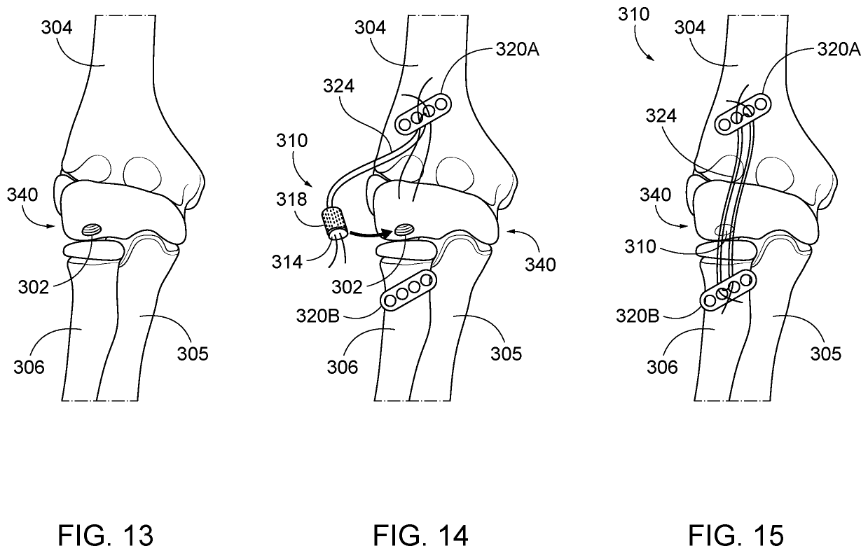

Referring to FIGS. 13-15, aspects of the invention relate to methods of repairing a ruptured or torn cartilage 302, at a repair site 340, involving a repair device 310 comprising a scaffold 314 and a graft 318, one or more fixation devices 320, and one or more sutures 324. In some embodiments, the scaffold 314, the graft 318, and the one or more sutures 324 are inserted into the repair site 1340 of the ruptured or torn cartilage 302 via arthroscopic equipment 230 (not depicted).

The repair site 340 is the area around a ruptured or torn cartilage 302 into which a device may be inserted. The repair device 310 may be inserted into the repair site 340 during surgery via the arthroscopic equipment 230 using techniques known to those of ordinary skill in the art. The scaffold 314 is expandable and can either fill the repair site 340 with the graft 318 or partially fill the repair site 340 with the graft 318. The scaffold 314 can partially fill the repair site 340 when inserted and expand to fill the repair site 340 in the presence of blood, plasma or other fluids either present within or added into the repair site 340.

An example of ruptured cartilage in the elbow is depicted in FIGS. 14-15. Elbow cartilage 302 is a crucial component within the joint that connects the humerus 304, to the radius 306 and ulna 305. Its primary role is to provide stability and reduce stress across the elbow joint. Elbow cartilage effectively restricts excessive forward or backward movement of the forearm bones in relation to the humerus 304, thereby maintaining joint alignment.

As shown in FIGS. 12 and 13, the elbow cartilage 302 is ruptured such that it forms a separation of cartilage in the capitellum of the humerus 304. Prior to insertion of the repair device 310, the affected extremity is prepared and draped in the standard sterile fashion. A tourniquet may be used if indicated. The ruptured cartilage 302 is identified and defined, and the tissue ends are pretreated, either mechanically or chemically. The one or more sutures 324 is connected to one or more fixation devices 320.

Prior to entering the repair site 340, the arthroscopic equipment 230 attaches the one or more sutures 324 to the repair device 310. The repair device 310 is treated with a repair material. In one embodiment, the repair device 310 may also be pre-treated in antibiotic solution prior to implantation.

In the illustrated embodiment, the one or more sutures 324 is then connected to the radius at the first end 326 via a first fixation device 320A. The first fixation device 320A is passed through the radius 306, carrying the one or more sutures 324. The first fixation device 320A and the one or more sutures 324 are attached to the radius 306. In one embodiment, the one or more sutures 324 may be further connected to the humerus 304 at the second end 328 via a second fixation device 320B.

The arthroscopic equipment 230 (not depicted) positions the repair device 310 along the one or more sutures 324 through the ruptured portion of the cartilage 302. In alternative embodiments, the arthroscopic equipment 230 positions the repair device 310 directly or indirectly onto the humerus 304 and/or the radius 306. The present disclosure may be used by insertion through an open incision. The repair device is compressible to allow introduction through arthroscopic portals, incisions and equipment.

The repair device 310 is then bonded to the surrounding tissue using the methods described herein. This can be done by the addition of a chemical agent or a physical agent such ultraviolet light, a laser, or heat. The repair device 310 may be reinforced by placement of additional sutures or clips. The arthroscopic portals is closed, and a sterile dressing placed. The post-operative rehabilitation is dependent on the type and size of lesion treated, and the tissue involved.

In the present disclosure, a subject includes, but is not limited to, any mammal, such as human, non-human primate, mouse, rat, dog, cat, horse or cow. In certain embodiments, a subject is a human. The present disclosure may also include kits for repair of ruptured or torn ligaments. A kit may include a scaffold of the invention having at least one fixation device attached to the scaffold and instructions for use. The scaffold may further include one or more sutures that attach an fixation device to the scaffold. A kit may further include a container that contains a repair material as described herein.

The foregoing written specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize or be able to ascertain using no more than routine experimentation, many equivalents to the specific embodiments of the invention described herein. Such equivalents are intended to be encompassed by the following claims.

What is claimed:

1. A tissue repair system, comprising:
a repair device including:
an implant sized and shaped for placement in a repair site of a torn tissue, the implant being compressible and expandable and configured to absorb a repair material, and
a graft configured to be coupled to the implant;
at least one implant suture configured to position the implant along or adjacent to a ruptured end of the torn tissue;
a first fixation device configured to couple the at least one implant suture to a first bone;
at least one graft suture configured to position the graft along or adjacent to the implant or the ruptured end of the torn ligament; and
a second fixation device configured to couple the at least one graft suture to the first bone.

2. The tissue repair system according to claim 1, wherein the torn tissue is a ligament.

3. The tissue repair system of claim 2, wherein the ligament is an ACL.

4. The tissue repair system according to claim 1, wherein the torn tissue is a tendon.

5. The tissue repair system according to claim 1, wherein the torn tissue is cartilage.

6. The tissue repair system according to claim 1, wherein the implant is a collagen scaffold configured to allow cell ingrowth.

7. The tissue repair system according to claim 1, wherein the graft is configured to be placed adjacent to the implant along a length of the implant.

8. The tissue repair system according to claim 1, wherein the graft is configured to wrap around the scaffold.

9. The tissue repair system according to claim 1, wherein the first fixation device is configured for placement against the first bone.

10. The tissue repair system according to claim 1, wherein the second fixation device is configured for placement against a second bone.

11. The tissue repair system according to claim 1, wherein the graft is selected from the group consisting of an autologous tendon graft, an allograft, and a synthetic tendon graft.

12. The tissue repair system according to claim 1, wherein the repair device and the at least one first suture is preloaded and insertable through an arthroscopic cannula.

13. The tissue repair system of claim 1, wherein the first fixation device and the second fixation device are selected from the group consisting of a screw, a barb, a helical fixation device, a staple, a clip, a snap, and a rivet.

14. The tissue repair system of claim 1, wherein the repair device further comprises a repair material.

15. The tissue repair system of claim 14, wherein the repair material is a platelet or plasma.

16. A method for repairing a ruptured tissue, comprising:
inserting a repair device comprising a scaffold and a graft proximate a ruptured end of the torn tissue of a patient; and
securing the scaffold to a suture and a first bone with a first fixation device.

17. The method of claim 16, further comprising coupling the repair device to a second bone with a second fixation device.

18. The method of claim 17, further comprising positioning the repair device between a ruptured end of a ligament and a femur and attaching the first fixation device to the femur and the second fixation device to the tibia.

19. The method of claim 17, further comprising positioning the repair device between a ruptured portion of cartilage.

20. The method of claim 16, further comprising positioning the repair device between a ruptured end of a tendon and a calcaneus bone and attaching the first fixation device to the calcaneus bone.

21. The method of claim 16, further comprising attaching the first fixation device only indirectly to the repair device.

22. The method of claim 16, wherein the scaffold comprises a porous collagen sponge.

23. The method of claim 16, further comprising treating the scaffold with a repair material.

24. The method of claim 16, further comprising:
harvesting a portion of a hamstring from the patient, and
forming the graft from the portion of the hamstring.

25. The method of claim 16, further comprising:
harvesting a portion of a hamstring from another patient, and
forming the graft from the portion of the hamstring.

26. The method of claim 16, further comprising:
assembling a bundle of synthetic polymer having a longitudinal dimension into the graft.
